(19) United States
(12) Patent Application Publication
(10) Pub. No.: US 2026/0346481 A1
(43) Pub. Date: Nov. 5, 2026

(54) CURVED TELESCOPIC DEVICE

(71) Applicant: KOREA INSTITUTE OF MEDICAL MICROROBOTICS, Gwangju (KR)

(72) Inventors: Jong Oh PARK, Gyeonggi-do (KR); Ja Young KIM, Daejeon (KR); Dohun LEE, Gwangju (KR); Kim Tien NGUYEN, Gwangju (KR)

(21) Appl. No.: 19/487,789

(22) PCT Filed: Oct. 25, 2023
(86) PCT No.: PCT/KR2023/016646

§ 371 Date: Nov. 25, 2025

(30) Foreign Application Priority Data
Aug. 16, 2023 (KR) 10-2023-0107188

(51) Int. Cl. B25J 18/02; F16M 13/02

(52) U.S. Cl. CPC B25J 18/025; F16M 13/022

(57) ABSTRACT

The present invention includes: N sliding shells, each of which has one end extending or retracting in an arc shape and is formed in a curved telescopic structure extendable or retractable in a curved shape; and a flexible member inserted into the N sliding shells, and which pushes or pulls at least one of the N sliding shells so as to extend or retract the N sliding shells.

### TECHNICAL FIELD

The present disclosure has been made with the support of the ministry of Science and ICT, the ministry of Trade, Industry and Energy, the ministry of Health and Welfare, and the Ministry of Food and Drug Safety of the Republic of Korea under Project ID. No. RS-2021-KD000001, and sub-project No. 1415184155, which was conducted by the Korea Institute of Medical Microrobotics in the research program named “microcarrier-based active precision delivery medical device development for regenerating knee cartilage” as a branch of the research project titled “Korea Medical Device Development Project (R&D)” under the research management of the Korea Medical Device Development Fund, from Jan. 1, 2023 to Dec. 31, 2023.

The present invention relates to a telescopic device, and more particularly, to a telescopic device using a curved telescopic mechanism applicable to medical fields (treatment, surgery, examination, etc.).

### BACKGROUND ART

Telescopic mechanisms are composed of overlapping or sliding components that may move relative to each other. The telescopic mechanisms have been used for various purposes throughout the industrial field since they allow long-stroke linear motion while maintaining a compact structure when folded. Examples of telescopic mechanisms include antennas, hydraulic cylinders, and the like.

Since the existing telescopic mechanisms could only perform linear motion, there was a limitation that they could be used only for lifting motion against gravity or simply linear motion moving back and forth. As such, since the existing telescopic mechanisms perform only linear operations in a limited manner, the field of application is limited.

A curved rail system includes a curved rail including an arc, a spiral curve, or a complex curve, and a carriage in which wheels, rollers, or bearings are mounted to smoothly move along the curved rail. The curved rail structure has excellent mobility flexibility and is widely used in applications such as camera equipment, robot arms, or material handling systems.

The internal and external human body structures, including the skeletal system, have a curved shape because the straight structure is vulnerable to impact, and the current medical equipment is primarily moving on a curved rail so as to move along the curve of the human body. A representative example of a curved rail structure is a C-arm structure of an X-ray image intensifier.

When using a curved rail structure, there are limitations in that it is difficult to miniaturize the device because the size of the medical device must be adjusted to the curved radius of driving, limiting the radius of the medical personnel's external field of view, and reducing the scope of medical personnel's intervention.

In this regard, Korean Patent Registration No. 10-2020149 discloses a telescopic 6-axis vertical articulated robot capable of selectively expanding and reducing the work area of the robot so that various tasks can be performed with one robot. The prior document may expand or reduce the work area of the robot through the first telescopic link and the second telescopic link, thereby performing various tasks. However, the prior document still uses a linear telescopic mechanism, and since it is not in a curved shape, it includes the problem of a linear telescopic structure as it is.

Accordingly, there is a need for a curved telescopic device to which a telescopic mechanism is applied while adopting a curved structure so as to have both advantages of a telescopic structure and a curved rail structure.

### DISCLOSURE OF INVENTION

### Technical Problem

An object of the present invention is to provide a curved telescopic device to which a telescopic mechanism is applied while adopting a curved structure so as to have both advantages of a telescopic structure and a curved rail structure.

### Solution to Problem

In order to achieve the above object, the present invention includes: N sliding shells having one end extending or retracting in an arc shape and formed in a curved telescopic structure that can be extended or retracted in a curved shape; and a flexible member inserted into the N sliding shells, and pushing out or pulling out at least one of the N sliding shells to extend or retract the N sliding shells.

Preferably, the N sliding shells may have one end to which an end effector is connected, and the other end to which a tilting part that enables fixing or tilting may be connected.

Preferably, the N sliding shells may further include a flexible member guide part provided for each sliding shell and preventing separation of the flexible member.

Preferably, the N sliding shells may include one or more bearings provided in at least one sliding shell.

Preferably, the N sliding shells may include: a rail provided on at least one surface of each sliding shell to prevent path deviation; and a limit block for preventing the sliding shell from being sucked into the adjacent sliding shell during a shortening process.

Preferably, the flexible member may be formed to be longer than a length when the N sliding shells are completely extended, and a remaining portion without being coupled to the N sliding shells may be located in a guide groove provided inside any one of the N sliding shells.

Preferably, the flexible member may be formed of a spring steel material.

Preferably, the flexible member may be connected to an outermost sliding shell among the N sliding shells to sequentially extend or shorten the N sliding shells.

Preferably, the device may further include a driving unit configured to push the flexible member to extend the N sliding shells, or to pull the flexible member to shorten the N sliding shells.

The driving unit may further include a motor, a first gear wheel connected to the motor to receive a driving force, and a second gear wheel engaged with the first gear wheel to receive a driving force and connected to a tensioner to adjust a distance from a pulley.

Preferably, the flexible member may include a plurality of tooth grooves capable of engaging with the teeth of the second gear wheel, and may push or pull the N sliding shells by repeating the operation of engaging with the tooth grooves by the rotation of the second gear wheel.

### Advantageous Effects of Invention

The present invention has an advantage in that it is possible to secure a space that an external mechanical structure, a camera, or a person can complementarily access in a compact folded state.

In addition, the present invention has the advantage of being able to easily access an area that is difficult to reach in the body by using the curved sliding shell.

In addition, the present invention is applied to a medical device, thereby improving the accuracy and stability of a medical procedure, solving the inconvenience of a patient, and improving the result.

### BRIEF DESCRIPTION OF THE DRAWINGS

FIG. 1 is a configuration diagram of a curved telescopic device according to an embodiment of the present invention.

FIG. 2 illustrates a folded curved telescopic device according to an embodiment of the present invention.

FIG. 3 illustrates an unfolded curved telescopic device according to an embodiment of the present invention.

FIG. 4 is N sliding shells according to an embodiment of the present invention, FIG. 4A shows a bottom view, and FIG. 4B shows a plan view.

FIG. 5 is a flexible member according to an embodiment of the present invention, FIG. 5A shows an unfolded state, and FIG. 5B shows a folded state.

FIG. 6 is a configuration diagram of a driving unit according to an embodiment of the present invention, FIG. 6A shows an exploded view, and FIG. 6B shows an assembled view.

FIG. 7 shows knee treatment equipment having three degrees of freedom using a magnetic field device as an end effector according to an embodiment of the present invention.

FIG. 8 shows brain surgical equipment having three degrees of freedom using a magnetic field device as an end effector according to an embodiment of the present invention.

### BEST MODE FOR CARRYING OUT THE INVENTION

A curved telescopic device, comprising:
N sliding shells having one end extending or retracting in an arc shape and formed in a curved telescopic structure extendable or retractable in a curved shape; and
a flexible member inserted into the N sliding shells and configured to push or pull at least one of the N sliding shells to extend or retract the N sliding shells.

### Description of the Preferred Embodiments

Hereinafter, the present invention will be described in detail with reference to the contents described in the accompanying drawings. However, the present disclosure is not limited by the exemplary embodiments. Like reference numerals presented in each drawing denote members that perform substantially the same function.

The objects and effects of the present disclosure may be naturally understood or become clearer by the following description, and the objects and effects of the present disclosure are not limited only by the following description. In addition, in describing the present disclosure, when it is determined that the detailed description of the known technology related to the present disclosure may unnecessarily obscure the gist of the present disclosure, the detailed description thereof will be omitted.

The terms used in the present invention are used only to describe specific embodiments, and are not intended to limit the present invention. Singular expressions include plural expressions unless the context clearly indicates otherwise. In the present application, it is to be understood that the terms such as “include” or “have” are intended to designate the presence of features, numbers, steps, operations, components, parts, or a combination thereof described in the description of the invention, and do not preclude the possibility of presence or addition of one or more other features, numbers, steps, operations, components, parts, or a combination thereof.

Terms such as first, second, etc. may be used to describe various components, but the components should not be limited by the terms. The terms are used only for the purpose of distinguishing one component from another. For example, without departing from the scope of the present disclosure, a first component may be referred to as a second component, and similarly, a second component may also be referred to as a first component.

Unless otherwise defined, all terms used herein, including technical or scientific terms, have the same meaning as those generally understood by those of ordinary skill in the art to which the present invention pertains. Terms such as those defined in commonly used dictionaries should be interpreted as having a meaning consistent with the meaning in the context of the related art, and unless clearly defined in the present invention, they are not interpreted as ideal or excessively formal meanings.

In interpreting the components, it is interpreted as including the error range even if there is no separate explicit description. The description of the temporal relationship includes, for example, a case in which the temporal precedence relationship is described as ‘after’, ‘following’, ‘thereafter’, ‘before’, etc., and a case in which the temporal precedence relationship is not continuous unless ‘immediately’ or ‘directly’ is used.

Hereinafter, the technical configuration of the present invention will be described in detail with reference to the accompanying drawings.

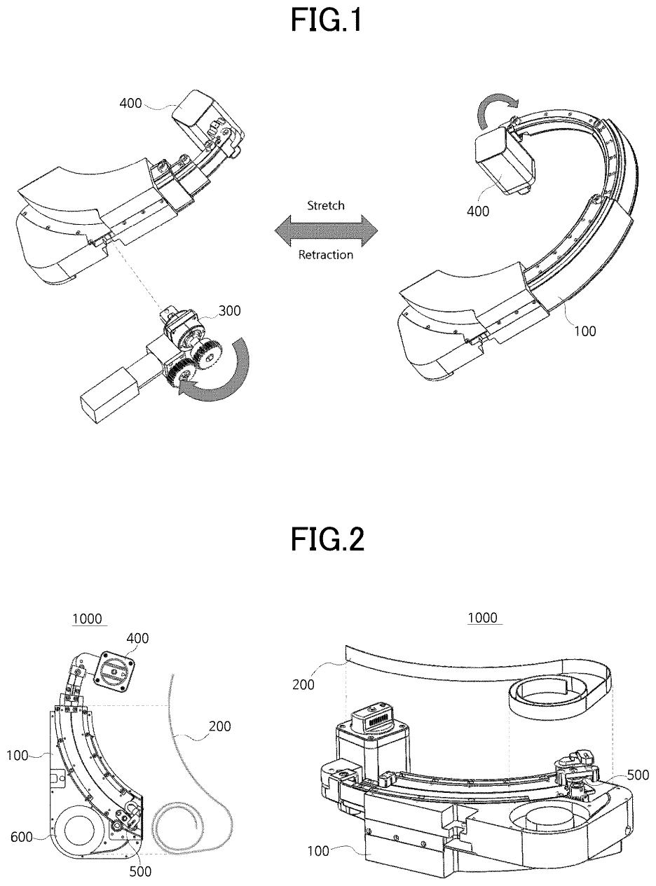

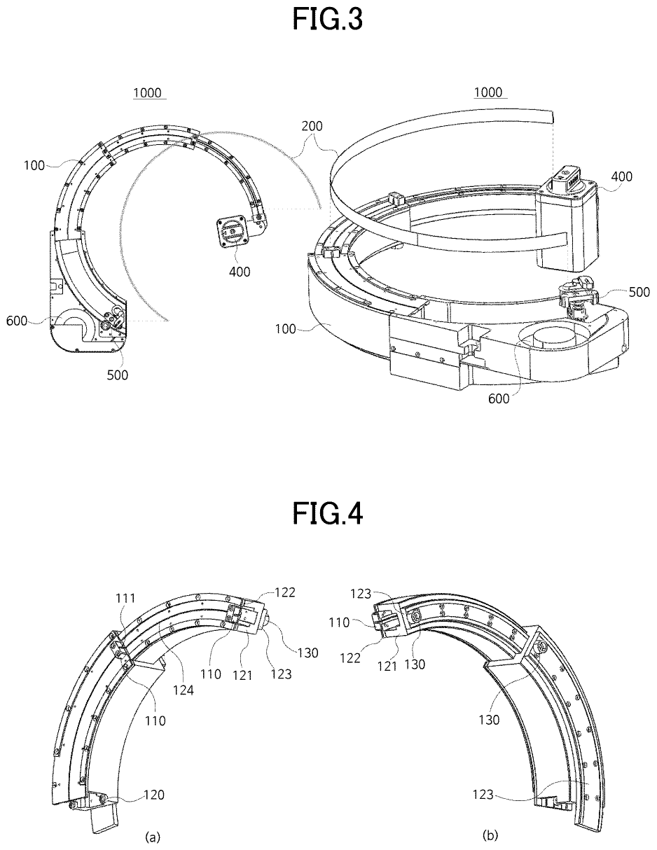

FIG. 1 is a configuration diagram of a curved telescopic device 1000 according to an embodiment of the present invention. FIG. 2 illustrates a folded curved telescopic device 1000 according to an embodiment of the present invention. FIG. 3 illustrates an unfolded curved telescopic device 1000 according to an embodiment of the present invention. Referring to FIGS. 1 to 3, a curved telescopic device 1000 may include N sliding shells 100, a flexible member 200, a tilting part 300, an end effector 400, a driving unit 500, and a guide groove 600. In the curved telescopic device 1000, a telescopic mechanism may be applied while adopting a curved structure so as to have both the advantages of a telescopic structure and a curved rail structure.

The curved telescopic device 1000 may be freely stretched or contracted as needed. Accordingly, the curved telescopic device 1000 may secure a space that may be complementarily accessed by an external mechanical structure, a camera, or a person in a compactly folded state. The curved telescopic device 1000 may be applied to the medical field to promote collaboration between a surgical robot and a medical expert, and may broaden the scope of observation and confirmation. The curved telescopic device 1000 has excellent visualization performance by allowing the imaging device to reach a region that is difficult to reach in the body through a curved telescopic mechanism.

Since the sliding shells move in a curved shape, the curved telescopic device 1000 may remove elements that may interfere with the field of view, thereby helping medical professionals in medical practice. The medical expert may perform the procedure more precisely and accurately using the curved telescopic device 1000 according to an embodiment of the present invention, thereby minimizing the patient's inconvenience and performing the optimal medical practice. The curved telescopic device 1000 may allow the imaging device to reach an area that is difficult to reach, thereby more clearly visualizing the target area to be observed and avoiding unintended contact with sensitive tissues, thereby reducing the risk of accidental injury or damage to surrounding tissues or structures.

Referring to FIG. 1, in the curved telescopic device 1000, N sliding shells may be sequentially connected, and an end effector may be connected to the innermost shell (movable shell). The curved telescopic device 1000 may be connected to a tilting part fixed and tiltable to the main body in the outermost shell (fixed shell). The entire N sliding shells can move smoothly by repeating the folded state and the unfolded state.

As shown in FIG. 1, the curved telescopic device 1000 has a large difference in the space occupied between the extension and the contraction. The curved telescopic device 1000 may maintain a contracted state when it is not necessary to use it to minimize the restriction of the behavior of the medical expert. The curved telescopic device 1000 may maintain an extended state in a situation in which use is required, and may adjust an extended length as necessary. That is, in the curved telescopic device 1000, when the end effector 400 is an imaging device, the N sliding shells 100 may be extended only to a desired portion to be photographed.

FIG. 4 is N sliding shells 100 according to an embodiment of the present invention, FIG. 4A shows a bottom view, and FIG. 4B shows a plan view.

The N sliding shells 100 may be formed in a curved telescopic structure in which one end is stretched or contracted in an arc shape and may be stretched or contracted in a curved shape. The conventional telescopic mechanisms have limitations in terms of space utilization and durability by adopting a linear structure. The N sliding shells 100 according to the present invention may overcome the limitations of the conventional straight telescopic mechanisms through the curved telescopic mechanisms.

The N sliding shells 100 may move smoothly by repeating a folded state and an unfolded state around the same radius. The N sliding shells 100 may be composed of a plurality of sliding shells, and preferably, the value of N is 2 or more. The value of N may be appropriately selected according to the environment used and the user's request, and is not limited to a specific value.

Hereinafter, in describing the present disclosure, when the value of N is 5, it may be understood that the sliding shells are arranged in the order of 1 to 5, the sliding shell 1 is a portion to which the end effector 400 is connected, and the sliding shell 5 is a portion to which the tilting part 300 or the driving unit 500 is connected.

The N sliding shells 100 may include a bottom module 121, a body module 122, and a rail 123. The outermost shell (fixed shell) of the N sliding shells 100 may not include the rail 123. The outermost shell (fixed shell) is fixed and does not include the rail 123 because it is not inserted into the other shell and contracted.

The bottom module 121 may be located below the body module 122 and may prevent the other sliding shell 100 inserted into the body module 122 from being separated. The bottom module 121 may be formed in a curved shape, and the radius of curvature may be the same as the body module 122 and the rail 123.

The bottom module 121 may include a first flexible member groove 124 and a flexible member guide part 110 at a lower portion thereof.

The flexible member guide part 110 has a second flexible member groove 111 connected to the first flexible member groove 124 formed therein. Since the second flexible member groove 111 is not completely open to the outside, the flexible member 200 is not separated to the outside. That is, the flexible member 200 is fixed between the first flexible member groove 124 and the second flexible member groove 111. A detailed description of the flexible member guide part 110 will be described again below.

The body module 122 may be positioned between the rail 123 and the bottom module 121. The body module 122 may have a space into which another sliding shell may be inserted. The inner space of the body module 122 may have an uneven shape ( [symbol] ). The uneven internal space may be divided into a large space and a narrow space based on a central horizontal line, and the narrow space may be a space through which a rail passes. The body module 122 may be formed in a curved shape, and the radius of curvature may be the same as the bottom module 121 and the rail 123.

The bearing 120 may be positioned at an end (a place in contact with the N+1 sliding shell) of the body module 122. When another sliding shell is inserted into the inner space of the body module 122, the bearing 120 may be positioned so as to be smoothly inserted with a low frictional force.

One end of the N sliding shells 100 may be connected to an end effector 400, and the other end thereof may be connected to a tilting part 300 that enables fixing or tilting.

The end effector 400 is a generic term for a suitable device installed at the end of the wrist of a robot that has only the wrist. The end effector 400 includes a tool (electric screwdriver, etc.), tongs, grippers, or the like. The end effector 400 according to an embodiment of the present invention may refer to an imaging device or a surgical tool that may be used by a medical expert. In addition, the above-described example of the end effector 400 is only an embodiment and is not limited thereto.

The end effector 400 is connected to one end of the N sliding shells 100 and has a joint structure, so that a predetermined angle may be adjusted up, down, left, and right, and may be configured to rotate 360 degrees with a pivot function.

The end effector 400 according to an embodiment of the present invention may move the same curved radius based on the center point according to the curved telescopic mechanism. Since the end effector 400 moves the same curved radius with respect to the center point, even if the position thereof is changed, it has a characteristic of exerting the same distance and influence toward the same direction (center point). Accordingly, it may be more effective when a magnetic device is used as the end effector 400.

The N sliding shells 100 may include a flexible member guide part 110, a bearing 120, a rail 123, and a limit block 130.

The flexible member guide part 110 may be provided for each sliding shell, and may prevent separation of the flexible member 200.

The flexible member guide part 110 has a second flexible member groove 111 connected to the first flexible member groove 124 formed therein. Since the second flexible member groove 111 is not completely open to the outside, the flexible member 200 is not separated to the outside. That is, the flexible member 200 is fixed between the first flexible member groove 124 and the second flexible member groove 111. The flexible member guide part 110 is provided only in the 1 to N−1-th sliding shell. That is, the outermost shell (fixed shell) among the N sliding shells 100 may not include the flexible member guide part 110. The outermost shell (fixed shell) has a driving unit 500 and may be fixed directly between the second gear wheel 530 and the pulley 550 without a separate flexible member guide part 110.

One or more bearings 120 may be provided in at least one sliding shell to reduce frictional force of the sliding shell in a process in which the sliding shell is stretched or contracted. Preferably, two bearings 120 may be provided. Since there are two places where a force acts in the process of moving the curved shell, two bearings 120 located at the end of the shell N−1 may be provided. However, this is only an embodiment and is not limited to the positions and the number of bearings mentioned above.

The rail 123 may be provided on at least one surface of each sliding shell and may prevent path deviation. In an embodiment, the rail 123 may be located at an upper end of the body module 122. The rail 123 may prevent the sliding shell from deviating from a path and vibration. The rail 123 may further improve the effect of preventing the sliding shell from deviating from the path by providing the limit block 130 at the upper portion thereof. The rail 123 may be formed in a curved shape, and the radius of curvature may be the same as the bottom module 121 and the body module 122.

The limit block 130 may prevent the sliding shell from being sucked into the adjacent sliding shell during the shortening process. The telescopic mechanisms are stretched or contracted in the process of one sliding shell being inserted into the other sliding shell and taken out again. In this case, when any one sliding shell is completely inserted into another sliding shell in a process of being inserted into the other sliding shell, difficulty may occur or failure may occur when the inserted sliding shell is taken out. Accordingly, the limit block 130 may be provided in the front portion of the sliding shell to prevent the sliding shell from being completely inserted into another sliding shell.

In an embodiment, the limit block 130 may be located above the rail 123 and may be formed in a circular shape. One limit block 130 may exist for each sliding shell. However, this is only an embodiment, and the position, shape, or number of limit blocks 130 is not limited thereto.

The flexible member 200 may be inserted into the N sliding shells 100, and may push out at least one of the N sliding shells 100 or pull out or retract the N sliding shells 100. The flexible member 200 may be used as a means for transmitting a driving force for stretching or contracting the sliding shell 100. That is, the flexible member 200 may push out the N sliding shells 100 or extend or contract the N sliding shells 100 by the driving force transmitted from the motor 510.

The flexible member 200 may extend or contract the N sliding shells 100 by using a change in length. At this time, the required length of the flexible member 200 is changed when the N sliding shells 100 are fully extended and fully contracted. Therefore, it may be a question of how to handle the difference in the length of the flexible member 200 in the fully extended state and the fully contracted state.

FIG. 5 is a flexible member 200 according to an embodiment of the present invention, FIG. 5A shows an unfolding state, and FIG. 5B shows a folding state. Referring to FIG. 5A, the flexible member 200 may form a semicircular shape in a fully extended state, that is, a fully unfolded state. In this case, since there is no significant difference between the length of the arc of the semicircle formed by the N sliding shells 100 and the length of the flexible member 200, handling of the remaining length is not a big problem.

Referring to FIG. 5B, the flexible member 200 is partially extended. In this case, the flexible member 200 may be used to extend and support the N sliding shells 100 by a length between A and B. The flexible member 200 may be located in the driving unit 500 by a length between B and C and may be used to receive a driving force. The length D in C of the flexible member 200 is a remaining length for additionally extending N sliding shells 100.

The flexible member 200 may be formed to be longer than the length when the N sliding shells 100 are fully extended, and the remaining portion without being coupled to the N sliding shells 100 may be located in the guide groove 600 provided inside any one of the N sliding shells 100.

Even after the N sliding shells 100 are fully stretched, the flexible member 200 may receive a driving force from the driving unit 500 to contract the N sliding shells 100. Accordingly, the flexible member 200 may be formed to be longer than the length of the half-circle arcs formed by the N sliding shells 100 that are completely extended and may extend to the driving unit 500.

As shown in FIG. 5, the flexible member 200 may be stored in the guide groove 600 by a remaining length (a length between C and D). In the process of stretching or contracting the N sliding shells 100, the remaining length of the flexible member 200 stored in the guide groove 600 may be flexibly changed. In a compact state in which the mode sliding shell is completely folded, the flexible member 200 may be rolled in a vortex shape along the guide groove 600.

The flexible member 200 may be connected to an outermost sliding shell (movable shell) among the N sliding shells 100 to sequentially extend or shorten the N sliding shells 100. That is, the shell actively moving by the driving force directly transmitted from the driving unit 500 may be a sliding shell (movable shell) located at the outermost side. The remaining sliding shells may be passively moved according to the movement of the sliding shell (movable shell) located at the outermost position. Each sliding shell has a step at the end, so that when a specific sliding shell in which another sliding shell is inserted is extended for a predetermined length or longer, it may no longer be extended and may be caught by the step.

As an example of the stretching process, assuming that there are five sliding shells, the first sliding shell connected to the flexible member 200 is pushed by the flexible member 200 in the stretching process, and when the first sliding shell is pushed to the end, the second sliding shell is pulled together by the movement of the first sliding shell. Similarly, when the second sliding shell is pushed to the end, the third sliding shell is pulled together by the movement of the second sliding shell, and when the third sliding shell is pushed to the end, the fourth sliding shell is pulled together by the movement of the third sliding shell. Since the fifth sliding shell is fixed, it is not pulled together by the fourth sliding shell. As such, the first sliding shell actively moves, and the second to fourth sliding shells passively move. In this way, the first slide shell to the fourth slide shell are sequentially stretched.

In an example of the contraction process, assuming that there are five sliding shells, the first sliding shell connected to the flexible member 200 in the contraction process is pulled by the flexible member 200, and when the first sliding shell is pulled to the end, the second sliding shell is pulled together by the movement of the first sliding shell. Similarly, when the second sliding shell is pulled to the end, the third sliding shell is pulled together by the movement of the second sliding shell, and when the third sliding shell is pulled to the end, the fourth sliding shell is pulled together by the movement of the third sliding shell. Since the fifth sliding shell is fixed, it is not pulled together by the fourth sliding shell. As such, the first sliding shell actively moves, and the second to fourth sliding shells passively move. In this way, the first slide shell to the fourth slide shell are sequentially shortened.

The flexible member 200 may be formed of a spring steel material. The flexible member 200 may be formed of a spring steel material having high strength, resistance to deformation, and the ability to return to its original shape. Preferably, the flexible member 200 may be formed of a spring steel plate. The material used as the flexible member 200 may have grades of 55, 65, 70, 75, 85, 65Mn, 60Si2Mn, and 50CrVA depending on the steel type.

The flexible member 200 may be inserted into the second flexible member guide part 110 in which the second flexible member groove 111 is formed. The flexible member 200 may not be separated to the outside due to the shape of the second flexible member groove 111 that is not completely open to the outside. That is, the flexible member 200 may be fixed between the first flexible member groove 124 and the second flexible member groove 111.

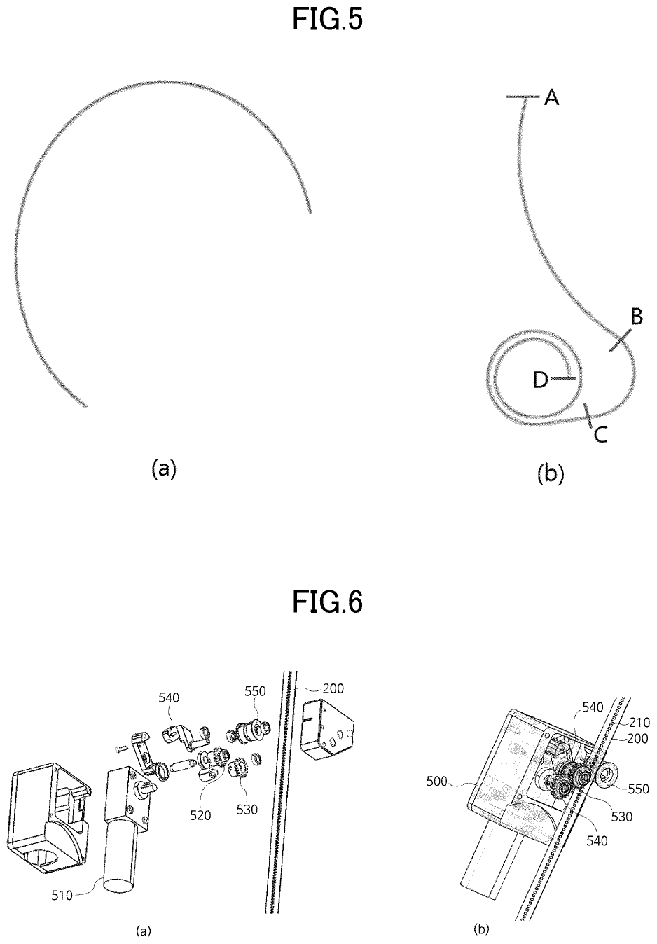

FIG. 6 is a configuration diagram of a driving unit according to an embodiment of the present invention, FIG. 6A shows an exploded view, and FIG. 6B shows an assembled view. Referring to FIGS. 6A and B, the driving unit 500 may include a motor 510, a first gear wheel 520, and a second gear wheel 530.

The driving unit 500 may push the flexible member 200 to extend the N sliding shells 100, or the N sliding shells 100 may pull the flexible member 200 to shorten the sliding shells.

Referring to FIG. 6A, the motor 510 may be connected to the first gear wheel 520. The motor 510 may generate a rotational force that rotates 360 degrees, and may transmit the generated rotational force to the first gear wheel 520 to rotate the first gear wheel 520. The motor 510 may be stored in a case and protected from external impact. The case may be fixed to an outermost shell (fixed shell).

The motor 510 may be configured as a high-torque motor, and may secure sufficient power to push or pull the N sliding shells 100 by using a dual gear.

The first gear wheel 520 may be connected to the motor 510 to receive a driving force. The teeth of the first gear wheel 520 may be engaged with the teeth of the second gear wheel 530. Accordingly, the first gear wheel 520 may transmit the rotational force received from the motor 510 to the second gear wheel 530 by engaging with the teeth of the first gear wheel 520, so that the second gear wheel 530 may rotate. As described above, the embodiment according to the present invention may secure sufficient power by using the dual gear.

The second gear wheel 530 may be engaged with the first gear wheel 520 to receive a driving force, and may be connected to a tensioner 540 to adjust a distance from the pulley 550.

The teeth of the second gear wheel 530 may be engaged with the teeth of the first gear wheel 520. Accordingly, the second gear wheel 530 may receive the rotational force from the motor 510 through the first gear wheel 520 by engaging with the teeth of the first gear wheel 520, and accordingly, the second gear wheel 530 may rotate. As described above, the embodiment according to the present invention may secure sufficient power by using the dual gear.

The flexible member 200 may include a plurality of tooth grooves 210 capable of being engaged with the teeth of the second gear wheel 530, and may push or pull the N sliding shells 100 by repeating the operation of being engaged with the tooth grooves 210 by the rotation of the second gear wheel.

The curved telescopic device 1000 according to an embodiment of the present invention may include a tensioner 540 and a tooth groove 210 to prevent backlash. Backless refers to a gap generated between gear teeth when a gear is turned in the opposite direction in a structure in which the teeth of another gear are engaged and rotated between the teeth of the gear. Backlash is an important factor in determining precision in precision machine control. A backlash is also properly needed to smoothly rotate a pair of gears. If there is too little backlash, lubrication is likely to be insufficient, and friction between teeth increases. In addition, if the backlash is too large, the engagement of the gear is deteriorated, and the gear is likely to be damaged.

The tensioner 540 may prevent backressing by adjusting a distance between the second gear wheel 530 and the pulley 550. In addition, since the tooth groove 210 is operated in engagement with the second gear wheel 530, backlashing may be prevented. In addition, the tooth groove 210 may be effectively used in calculating the position of the end effector through the encoder. For example, if the distance between the tooth grooves 210 is measured in advance, how many tooth grooves 210 have passed is calculated, and the position of the end effector 400 is known for the initial position of the end effector 400, the position of the end effector 400 may be easily calculated.

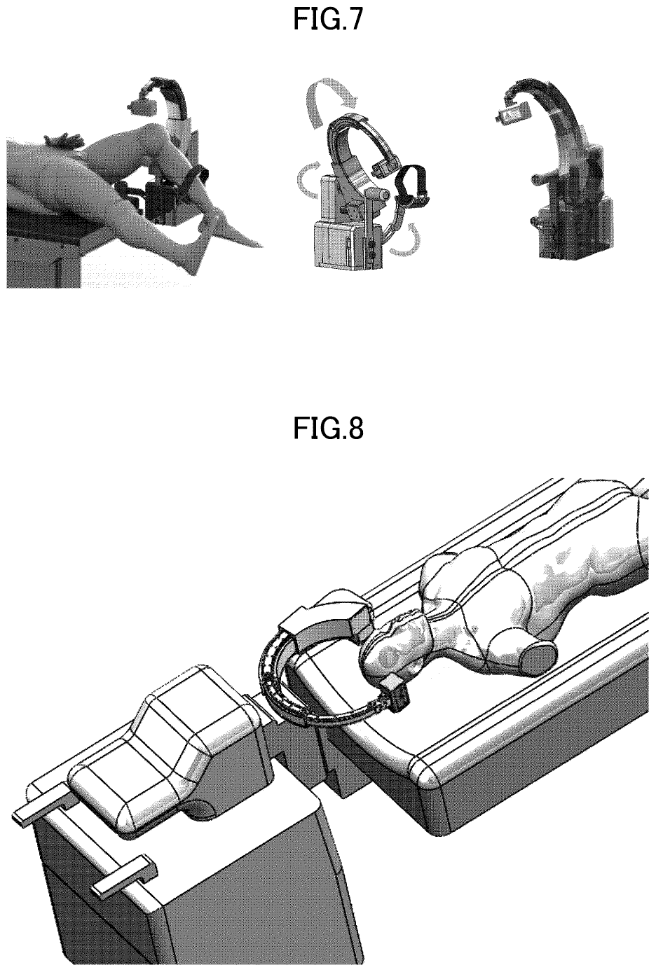

FIG. 7 shows knee treatment equipment having three degrees of freedom using a magnetic field device as an end effector according to an embodiment of the present invention. Referring to FIG. 7, the knee treatment equipment may include a fixing frame, a curved telescopic device 1000, and a leg holder.

The fixing frame can support the knee treatment equipment to stand on the floor. The fixing frame may be connected to the curved telescopic device 1000 through the tilting part 300. The fixing frame may be connected to the leg holder on a different axis from the curved telescopic device 1000.

The curved telescopic device 1000 used in the knee treatment device is the same as described above. However, a magnetic field device may be used as the end effector 400. That is, the knee treatment device is advantageous for image visualization because it is possible to photograph a part that is generally difficult to photograph by using the curved telescopic device 1000 in which a magnetic device is used as the end effector 400.

The leg holder is the part where the patient's leg to be photographed is placed and can be moved up and down in an arc. The leg holder provides a comfortable environment to the patient being photographed, and fixes the knee so that photographing may be smoothly performed in the curved telescopic device 1000.

The knee treatment equipment may have a total of three degrees of freedom through rotation (1 degree of freedom) of the curved telescopic device 1000 through the tilting part 300, elongation and contraction (1 degree of freedom) of the N sliding shells, and vertical movement (1 degree of freedom) of the leg holder.

FIG. 8 shows brain surgical equipment having three degrees of freedom using a magnetic field device as an end effector according to an embodiment of the present invention. Referring to FIG. 8, the brain surgical equipment may include a bed, a curved telescopic device 1000, and a holder.

The bed may provide an environment in which the patient may lie down for brain surgery or imaging. A bed is a medical bed and can be collectively referred to as a bed generally used in an operating table. The bed may be connected to the curved telescopic device 1000 through the tilting part 300.

The curved telescopic device 1000 used in the brain surgical equipment is the same as described above. However, a magnetic field device may be used as the end effector 400. That is, the brain surgical equipment is advantageous for image visualization because it can capture even a part that is generally difficult to capture using the curved telescopic device 1000 in which the magnetic device is used as the end effector 400.

The holder may be disposed to hold the sliding shell portion of the curved telescopic device 1000 when the curved telescopic device 1000 crosses the bed.

Although the present invention has been described in detail through the exemplary embodiments above, those skilled in the art to which the present invention belongs will understand that various modifications are possible to the above-described embodiments within the scope of the present invention. Therefore, the scope of the present invention should not be limited to the described embodiments, but should be defined by all changes or modifications derived from the concept of patent claims and the concept of equality as well as the scope of patent claims to be described later.

[Description of Reference Numerals]

| | |
|---|---|
| 100: N sliding shells | 110: Flexible member guiding part |
| 111: Second flexible member groove | 120: Bearing |
| 121: Bottom module | 122: Body module |
| 123: Rail | 124: First flexible member groove |
| 130: Limit block | 200: Flexible member |
| 210: Tooth groove | 300: Tilting part |
| 400: End effector | 500: Driving unit |
| 510: Motor | 520: First gear wheel |
| 530: Second gear wheel | 540: Tensioner |
| 550: Pulley | 600: Guide groove |
| 1000: Curved telescopic device | |

### INDUSTRIAL APPLICABILITY

An object of the present invention is to provide a curved telescopic device to which a telescopic mechanism is applied while adopting a curved structure so as to have both advantages of a telescopic structure and a curved rail structure.

What is claimed is:

1. A curved telescopic device, comprising:
N sliding shells having one end extending or retracting in an arc shape and formed in a curved telescopic structure extendable or retractable in a curved shape; and
a flexible member inserted into the N sliding shells and configured to push or pull at least one of the N sliding shells to extend or retract the N sliding shells.

2. The curved telescopic device of claim 1, wherein
the N sliding shells have one end connected to an end effector, and the other end connected to a tilting part that is fixed or tilted.

3. The curved telescopic device of claim 1, wherein
the N sliding shells further comprise flexible member guide parts each provided in each sliding shell, and configured to prevent the flexible member from being separated.

4. The curved telescopic device of claim 1, wherein
the N sliding shells comprise at least one bearing provided on at least one sliding shell to reduce frictional force of the sliding shell in a process in which the sliding shell is stretched or contracted.

5. The curved telescopic device of claim 1, wherein
the N sliding shells comprise:
a rail provided on at least one surface of each sliding shell to prevent a path deviation; and
a limit block to prevent the sliding shell from being sucked into an adjacent sliding shell in a shortening process.

6. The curved telescopic device of claim 1, wherein
the flexible member is formed to be longer than a length when the N sliding shells are fully extended, and a remaining portion without being coupled to the N sliding shells is located in a guide groove provided inside any one of the N sliding shells.

7. The curved telescopic device of claim 1, wherein
the flexible member is formed of a spring steel material.

8. The curved telescopic device of claim 1, wherein
the flexible member is connected to an outermost sliding shell among the N sliding shells to sequentially extend or shorten the N sliding shells.

9. The curved telescopic device of claim 1, further comprising:
a driving unit configured to push the flexible member to extend the N sliding shells, or to pull the flexible member to shorten the N sliding shells.

10. The curved telescopic device of claim 9, wherein
the driving unit comprises:
a motor;
a first gear wheel connected to the motor to receive a driving force; and
a second gear wheel engaged with the first gear wheel to receive a driving force, and connected to a tensioner to adjust a distance from a pulley.

11. The curved telescopic device of claim 10, wherein
the flexible member comprises a plurality of teeth grooves capable of engaging with the teeth of the second gear wheel, and pushes or pulls the N sliding shells by repeating the operation of engaging with the teeth grooves by the rotation of the second gear wheel.
